(19) United States
(12) Patent Application Publication
(10) Pub. No.: US 2023/0356006 A1
(43) Pub. Date: Nov. 9, 2023

(54) APPLICATION OF PRESSURE WAVES TO SKIN AND DEEPER ORGANS

(71) Applicant: Moshe Ein-Gal, Ramat Hasharon (IL)

(72) Inventor: Moshe Ein-Gal, Ramat Hasharon (IL)

(21) Appl. No.: 18/353,202

(22) Filed: Jul. 17, 2023

Related U.S. Application Data
(63) Continuation-in-part of application No. 17/665,743, filed on Feb. 7, 2022.

(51) Int. Cl. A61N 7/00

(52) U.S. Cl. CPC A61N 7/00; A61N 2007/0034

(57) ABSTRACT

A method and device for skin or deeper organ treatment uses an applicator that includes a transducer for producing beams of pressure waves and a coupler for coupling the pressure waves to the skin or deeper organ. The pressure waves in the skin and deeper propagate in directions generally extending at small angles relative to the skin surface

### CROSS-REFERENCE TO OTHER APPLICATIONS

This application claims priority from, and is a continuation-in-part of, U.S. patent application Ser. No. 17/665,743, filed Feb. 7, 2022, which is incorporated herein by reference.

### FIELD OF THE INVENTION

The present invention generally relates to methods and devices for application of pressure waves to skin and deeper organs for medical treatment.

### BACKGROUND OF THE INVENTION

Medical and cosmetic treatments have been developed that emit beams of pressure waves to perform a medical or cosmetic procedure in the skin and deeper organs. There is a central beam (along the mean direction of the beam) and a beam aperture (the contour of the wavefront exiting the applicator). A beam may be converging, diverging or generally parallel. The beam penetrates the skin by means of some type of an applicator's coupler-exit that contacts the skin.

The central beams penetrate the skin generally perpendicularly to the skin (and perpendicular to the coupler-exit). The physical parameters of the beam, such as intensity, frequency and temporal profile, are constrained by considerations of efficacy and safety related to the skin or target organ and to adjacent tissue.

U.S. patent Ser. No. 10/695,588 describes alopecia treatment by application of perpendicular beams of pressure waves crossing the coupler/scalp boundary. Depending on the beam's physical parameters, the waves may also cross the scalp/skull and skull/brain boundaries. Respective refraction and transmission take place in each boundary (e.g., at the external surface of the skull or scalp and at the internal boundary to the brain), and there is beam attenuation in between the respective boundaries.

Prevention of brain damage constrains the waves' physical parameters and may affect the treatment performance. Respective scalp and skull thickness, acoustic impedance and attenuation of the beam, affect determination of the physical beam's parameters. In general, subcutaneous tissue layers should be taken into account when treating skin regions even other than the scalp.

The coupler angle and the skin angle are defined as the angles between the respective central beam angles of coupler and skin relative to the perpendicular to the interface of the coupler-exit-surface and the skin. A generally rigid coupler has a fixed angle between the central coupler beam and the coupler exit surface.

Typical pressure wave transducers produce pressure waves using methods known as: a) electromagnetic, b) electrohydraulic, c) piezoelectric and ultrasonic or d) radial. The methods are respectively characterized by a) electromagnetic force causing a membrane to jerk, b) discharging a capacitor giving rise to a spark between two electrodes submerged in water, c) producing pressure waves by distorting crystals due to pulsed or harmonic electrical fields, and d) accelerating a small projectile so as to produce pressure waves due to the projectile impinging on a generally metallic body.

Apertures (wave front contours) related to pressure waves transducers and couplers are typically circular.

The coupler exit is a portion of the coupler surface configured to couple pressure waves from a transducer to the skin. A thin lubricating layer is typically used for improved skin contact.

### SUMMARY OF THE INVENTION

The present invention seeks to provide a method and a device for skin and deeper organs treatment, as is described more in detail below. The invention uses an applicator that includes a transducer for producing beams of pressure waves and a coupler for coupling the pressure waves to the skin. The pressure waves in the skin propagate in directions generally extending large skin angles, defined relative to the vertical (“vertical” meaning an imaginary axis normal to the skin surface).

For treatments of deeper organs, e.g., a kidney, the beam change of direction at the skin/coupler interface may assist to get around an obstacle, e.g., a rib.

There is provided in accordance with a non-limiting embodiment of the invention an applicator including at least one transducer configured to produce a pressure wave beam, and a coupler configured to couple the pressure wave beam to a skin via a coupler exit, wherein the coupler exit surface is tilted from the vertical axis at a coupler angle which is an acute angle less than 90°.

Beams can be time-wise continuous, pulsed or a mixture.

### BRIEF DESCRIPTION OF THE DRAWINGS

The present invention will be understood and appreciated more fully from the following detailed description taken in conjunction with the drawings in which:

FIG. 1 is a simplified pictorial illustration of a pressure wave applicator, in accordance with a non-limiting embodiment of the present invention. Although the description is for skin treatment, it also applies to treatment of deeper organs beneath the skin.

### DETAILED DESCRIPTION

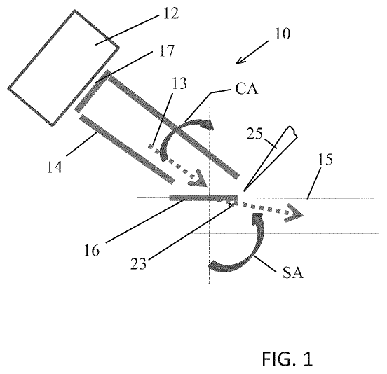

Reference is now made to FIG. 1, which illustrates a pressure wave applicator 10, in accordance with a non-limiting embodiment of the present invention.

Applicator 10 may include a transducer 12 (or an array of transducers 12) configured to produce a beam 13 of pressure waves. Non-limiting examples of suitable transducers 12 include electromagnetic, electrohydraulic, piezo-electric or radial transducers, or any combination thereof.

A coupler 14 is configured to couple the pressure wave beam 13 to a skin surface 15 via a coupler exit 16. The coupler 14 is tilted from the vertical at a coupler angle CA. The material of coupler 14 may be mostly liquid, such as, without limitation, water, or mostly elastomeric or solid, such as, without limitation, rubber or silicone.

The pressures waves are refracted and propagate in the skin at a skin angle SA, which is relatively large from the vertical (from the vertical SA>CA), and at a relatively small angle with respect to the skin surface, such as when the coupler propagation velocity is less than the skin propagation velocity. Using such a large skin angle reduces the waves' penetration depth to subcutaneous tissue layers and increases the skin volume subjected to a beam of pressure waves.

The acoustic propagation velocity of the coupler 14 is less than the acoustic propagation velocity of the skin 15, such as, without limitation, by at least 5%, alternatively by at least 15%, and further alternatively by at least 25%.

The coupler angle CA, which is the angle between the mean propagation direction of the pressure waves (the central axis) in the coupler 14 and the normal to the surface of the coupler exit 16, is an acute angle less than 90°, such as, without limitation, greater than 20°, alternatively greater than 40° and further alternatively greater than 60°, depending on the respective acoustic propagation velocitys of the skin 15 and the coupler 14 and the desired skin angle.

The applicator 10 has an aperture 17 at the exit of the transducer 12 (giving rise to the wavefront shape), which may be, without limitation, generally rectangular, square, circular, elliptical or matched to the treated skin surface.

The beam 13 at the coupler exit may be convergent, divergent or generally parallel. The coupler 14 may include a beam modifier, such as, without limitation, a lens or a reflector.

In one embodiment, the coupler angle CA may be determined according to the desired skin angle SA by selecting the respective acoustic propagation velocitys of the coupler and the skin (Snell's law):

Sin(coupler angle)=Sin(skin angle)×propagation velocity (coupler)/propagation velocity(skin)

In theory, a critical coupler angle (for skin angle=90°) is given by:

Sin(Coupler angle)=propagation velocity (coupler)/propagation velocity (skin).

A coupler angle close to the critical coupler angle may significantly reduce the transmitted energy; a transition from longitudinal waves to shear waves may also take place.

For scalp treatment for combatting alopecia, the coupler's propagation velocity and angle, may be selected so as to allow increasing rate of treated scalp volume while limiting the risk to the brain.

What is claimed is:

1. An applicator comprising:
at least one transducer configured to produce a pressure wave beam comprising a central beam defined as a beam along a mean direction of said pressure wave beam; and
a coupler configured to couple said pressure wave beam to a skin via a coupler exit surface attachable to the skin, wherein said coupler exit surface is tilted with respect to the central beam at a coupler angle which is an acute angle less than 90°.

2. The applicator according to claim 1, wherein an acoustic propagation velocity of said coupler is lower than an acoustic propagation velocity of the skin.

3. The applicator according to claim 2, wherein the acoustic propagation velocity of said coupler is lower than the acoustic propagation velocity of the skin by at least 5%.

4. The applicator according to claim 2, wherein the acoustic propagation velocity of said coupler is lower than the acoustic propagation velocity of the skin by at least 15%.

5. The applicator according to claim 2, wherein the acoustic propagation velocity of said coupler is lower than the acoustic propagation velocity of the skin by at least 25%.

6. The applicator according to claim 1, wherein the coupler angle is greater than 60°.

7. The applicator according to claim 1, wherein the coupler angle is greater than 45°.

8. The applicator according to claim 1, wherein the coupler angle is greater than 30°.

9. The applicator according to claim 1, wherein said at least one transducer comprises at least one of an electromagnetic transducer, an electrohydraulic transducer, a piezo-electric transducer, an ultrasonic transducer and a radial transducer.

10. The applicator according to claim 1, wherein an applicator aperture that gives rise to a wavefront shape of said beam is generally rectangular, square, circular or elliptical.

11. The applicator according to claim 1, wherein said beam at said coupler exit is convergent, divergent or generally parallel.

12. The applicator according to claim 1, wherein said coupler comprises a beam modifier.

13. A method for treating skin and deeper organs comprising using the applicator of claim 1 to treat the skin or tissue of deeper organs.

14. The method according to claim 13, wherein an acoustic propagation velocity of said coupler is lower than an acoustic propagation velocity of the skin or the tissue attachable to the coupler.
